(19) United States
(12) Patent Application Publication
(10) Pub. No.: US 2003/0165956 A1
(43) Pub. Date: Sep. 4, 2003

(54) Electrophoretic assay to predict risk of cancer and the efficacy and toxicity of cancer therapy

(75) Inventors: Craig W. Stevens, Houston, TX (US); Sheikh Ismail, Houston, TX (US); Thomas Buchholz, West University, TX (US); Michael Story, Houston, TX (US); William Brock, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/351,247

(22) Filed: Jan. 24, 2003

Related U.S. Application Data
(60) Provisional application No. 60/351,732, filed on Jan. 25, 2002.

(51) Int. Cl. C12Q 1/68

(52) U.S. Cl. 435/6

(57) ABSTRACT

The present invention provides a method for predicting the risk of occurrence of cancer. It also predicts the presence of BRCA mutations which in turn predicts the risk of developing breast cancer in women. Further, it assesses a cancer patient's level of sensitivity to chemotherapy.

This application claims priority to co-pending U.S. Provisional Application, Serial No. 60/351,732 filed Jan. 25, 2002. The entire text of the above-referenced disclosure is specifically incorporated herein by reference without disclaimer.

### BACKGROUND OF THE INVENTION

I. Field of the Invention

The present invention relates generally to the fields of molecular biology and oncology. More particularly, it concerns the use of electrophoretic mobility shift assays for the prediction of susceptibility of cells to DNA damage, prediction of toxicity from radiation and chemotherapy, the prediction of risk of occurrence of cancer in an individual, and the prediction of presence of BRCA mutations.

II. Description of Related Art

Approximately 1.2 million Americans are expected to develop cancer this year, and one patient in three will receive radiotherapy during the course of their disease. Since radiation complications occur in 5-10% of these patients, this means that 20,000 to 40,000 patients will suffer long term complications per year. This problem will become more serious as cancer survival increases. Radiation complications are dependent on the organ irradiated, the volume of that organ irradiated, how the radiation is delivered (daily dose and total dose), and the intrinsic radiosensitivity of the patient. Complications are not manifest in all patients at high risk, or may be manifest quite late after treatment. Late radiation complications are often modeled as a stochastic process, but can be affected by DNA repair problems.

Current radiation oncology practice guidelines assume that the risk of complications in an individual can be predicted by the complication rates seen in similar populations. However, this line of reasoning (which treats all patients the same regardless of risk) limits the dose that is delivered to relatively resistant patients while providing a relatively high risk of complications to others. Current radiation dose escalation trials propose doses as high as 102.9 Gy (Hayman et al., 2001), but paradoxically the maximum tolerated dose for the largest tumors was only 65.1 Gy. If the most radiosensitive patients could be identified, the remaining patients could possibly be escalated to higher doses. Similarly, techniques to reduce toxicity might be best applied to the most radiosensitive patients, since they would be at greatest risk for complications. In lung cancer, for example, this might involve the use of respiratory gating, using time consuming but more reliable patient set-up techniques, and perhaps the use of radioprotectors like amifostine. As more resource-intensive highly conformal therapies become available (like IMRT, proton therapy, etc.), these could be first applied to those patients at greatest risk of side effects.

It has been hypothesized that tumor radiocurability will be a function of the starting number of tumor cells, the intrinsic tumor cell radiosensitivity, tumor hypoxia, and tumor proliferation rate. There are currently good assays for most of these parameters. Tumor cell number can be estimated by measuring tumor size, perhaps with an estimate of cell viability based on FDG-PET scanning; tumor hypoxia can be estimated with several techniques such as Eppendorf microelectrode measurements or nitroimidazole binding; and tumor proliferation rate can be estimated by potential doubling time (Tpot). However, tumor cell radiosensitivity has been difficult to measure. While fibroblasts from any given patient would be expected to be very homogeneous, tumor cells would be expected to be quite variable because of their inherent genetic instability. Since the plating efficiency of tumor cells derived from patients is so poor, it might be expected that some significant populations might be under-represented when placed under such strong selection. Also, because of tumor heterogeneity, there may be uncommon subsets of tumor cells that, if very radioresistant, may ultimately determine outcome. Sampling error could omit these cells entirely from the biopsy.

One approach in predicting tumor radiosensitivity is based on a more comprehensive analysis of protein differences between radiosensitive and resistant cells to developed a predictive assay. cDNA microarray technology has been applied to the study of radiation resistance. A recent paper (Achary et al., 2000) describes the comparison of the expression profile of two cervical tumor cell lines derived from the same patient—one cell line being relatively more sensitive to radiation than the other. In this study the expression of 5776 genes (of an estimated 80,000 that human cells theoretically can express) were analyzed, and 52 sequences found to be elevated in the resistant cell line and 18 in the sensitive cell line. Most of these genes were either unknown, or not previously thought to be relevant to DNA repair. Although, this type of analysis may improve the understanding of global gene expression it would miss any radiosensitive cells whose mechanism of radiosensitivity was independent of gene expression (e.g., mutations in active sites, splice variants, mutations that affected protein stability or transport to the nucleus, or post-translational modification). Similarly, although proteomics may help to overcome some of these drawbacks, it will not be beneficial to others.

Much work has been done to minimize toxicity from sources over which the radiation oncologist has control, but tests for intrinsic radiosensitivity have also been problematic. Clonogenic survival has been correlated with outcome and toxicity, but clonogenic assays often take months to complete and are very expensive.

Thus, predicting normal tissue and tumor radiosensitivity have been desirable, but elusive, goals in radiobiology (Peters and McKay, 2001). There is currently no clinically useful test to predict radiosensitivity of a patient's normal tissue or tumor. It would therefore be very helpful to have an intermediate marker that can determine the optimal dose of a biologic agent, and the time at which the radiosensitizing effect (if any, for that tumor) is maximal within an individual tumor.

At present, there is no simple and cost effective way to determine radiosensitivity of tumor or normal tissue. The best current approach involves growing tumor and normal tissue from each patient (a process that takes many weeks), followed by measuring the surviving fraction (SF2) of cells after 2Gy of irradiation (a process that takes several more weeks). However, this is expected to cost over $5000.00 per sample. Thus, new useful and inexpensive screening assays are needed.

### SUMMARY OF THE INVENTION

The present invention overcomes these and other defects in the art and provides methods to assess the susceptibility of cells to DNA damage. It also provides methods for predicting the risk of cancer in an individual, predicting the presence of BRCA mutations in an individual, predicting the toxicity of a DNA damaging cancer therapy in an individual and measuring tumor cell sensitivity to a DNA damaging cancer therapy in an individual.

Thus, in accordance with the present invention, there is provided a method for assessing the susceptibility of a cell to DNA damage comprising the steps of providing an extract comprising proteins from a cell, mixing the extract with a labeled oligonucleotide and an excess of non-labeled DNA, subjecting the above mixture to electrophoretic separation, determining the band shift of said labeled oligonucleotide and comparing the band shift of said labeled oligonucleotide with that observed when a control is used. The resultant change in band shift indicates an altered sensitivity to DNA damage.

In some embodiments of the method, the proteinaceous extract obtained from the cell is a nuclear protein extract. The cell from which the protein extract is obtained may be a primary fibroblast cell or a lymphocyte. In some embodiments of the method, the cell may be obtained from a blood or a tissue sample. The sample may be from a subject that has cancer, or a subject that does not have cancer.

In some embodiments of the method, the oligonucleotide is end-labeled. In yet further embodiments, the label may be a radiolabel, a fluorescence label, a dye or an enzyme. In a particular embodiment of the method, the label is ³²P.

In some embodiments of the method, the non-labeled DNA is supercoiled DNA. In some embodiments of the invention, the control may comprise proteins from a radiosensitive and/or a non-radiosensitive cell.

In certain embodiments, the susceptibility of the cells is to DNA damage by radiation. The radiation source may be ionizing radiations such as x-irradiation and/or gamma-irradiation. In other embodiments, the susceptibility of cells is to chemical DNA damage, such as from cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate.

The present invention also provides a method for predicting the risk of cancer in an individual. The method comprises the steps of providing an extract comprising proteins from a cell, mixing the extract with a labeled oligonucleotide and an excess of non-labeled DNA, subjecting the above mixture to electrophoretic separation, determining the band shift of said labeled oligonucleotide and comparing the band shift of said labeled oligonucleotide with that observed when a control is used. The resultant change in band shift indicates an altered sensitivity to DNA damage which predicts altered risk of cancer in an individual.

The present invention also provides a method for predicting the presence of BRCA mutations in an individual. The method comprises the steps of providing an extract comprising proteins from a cell, mixing the extract with a labeled oligonucleotide and an excess of non-labeled DNA, subjecting the above mixture to electrophoretic separation, determining the band shift of said labeled oligonucleotide and comparing the band shift of said labeled oligonucleotide with that observed when a control is used. The decrease in band shift indicates BRCA mutation damage, which predicts an increased risk of breast cancer in said individual.

The present invention further provides a method for predicting the toxicity of a DNA damaging cancer therapy. The method comprises the steps of providing an extract comprising proteins from a cell, mixing the extract with a labeled oligonucleotide and an excess of non-labeled DNA, subjecting the above mixture to electrophoretic separation, determining the band shift of said labeled oligonucleotide and comparing the band shift of said labeled oligonucleotide with that observed when a control is used. The relative decrease in band shift indicates relative therapeutic toxicity.

The present invention further provides a method for measuring tumor cell sensitivity to a DNA damaging cancer therapy. The method comprises the steps of providing an extract comprising proteins from a cell, mixing the extract with a labeled oligonucleotide and an excess of non-labeled DNA, subjecting the above mixture to electrophoretic separation, determining the band shift of said labeled oligonucleotide and comparing the band shift of said labeled oligonucleotide with that observed when a control is used. The decrease in band shift is indicative of cells being sensitive to the DNA damaging cancer therapy.

In addition, the present invention provides methods of treating cancer, performed in conjunction with each of the methods described above. Such therapeutic methods include radio-, chemo-, immuno- or gene therapy. Cancers suitable for prediction or treatment include breast cancer, lung cancer, brain cancer, pancreatic cancer, ovarian cancer, cervical cancer, testicular cancer, stomach cancer, colon cancer, head & neck cancer, liver cancer, melanoma, leukemia, esophageal cancer or uterine cancer.

“A” or “an” is defined herein to mean one or more than one. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Electrophoretic Mobility Shift Assay (EMSA).

FIG. 2. EMSA of human BRCA heterozygotes and controls.

FIG. 3A. Densitometry on Complex A.

FIG. 3B. Complex A density vs. SF2.

FIG. 4. Eluted DNA End Binding Complexes (EBCs).

FIG. 5. Protein Identification of Supershifts.

FIG. 6. Western blot analysis of purified EMSA complexes to confirm the presence of individual proteins

FIG. 7. Comparison of normal mouse and human EMSA banding patterns.

FIG. 8. Anti-ATM antibodies supershift of all of the bands absent in AT cells.

FIG. 9. Band A density correlates with SF2. This is a composite of all primary fibroblast data. The correlation coefficient for linear regression was 0.75, for polynomial regression (as plotted) the correlation coefficient was 0.82.

FIG. 10. Comparison of DNA-EBC pattern from fibroblasts (lanes 1 and 2) and lymphocytes from unrelated individuals (lanes 3-6).

FIG. 11. DNA-EBC pattern of lymphocytes (lane 1) and fibroblasts (lane 2) from a patient heterozygous for ATM mutation, compared to C29 normal.

FIG. 12. Effects of SC236 (SC) on DNA-EBC pattern relative to untreated cells (C) for 2 tumor cell lines.

FIG. 13. The histone deacetylase inhibitor sodium butyrate radiosensitizes, and this is predicted by DNA-EBC.

FIG. 14. The radiosensitizing effects of MDA-7 gene therapy on A549 lung carcinoma cells are predicted by DNA-EBC.

FIG. 15. Results of mixing nuclear extracts. (Panel A) Band A density of AT cells (which is very low) is stable until it is contaminated by more than 20% with proteins from resistant human cells. (Panel B) Band A density of C80 cell extracts is stable when less than 10% contaminated with rodent proteins. (Panel C) Rodent nuclear extracts do not affect the band A density of AT cell extracts.

FIG. 16. Supershift analysis of DNA-EBCs from normal human cells using antibodies to the indicated protein. Panels A, B, and C demonstrate positive supershift results, which show that the indicated protein is present in band A.

FIG. 17. Supershift analysis of DNA-EBCs from normal human cells using antibodies to the indicated protein. None of the antibodies resulted in a supershift of band A.

FIG. 18. Western analysis of band A components in primary fibroblasts from patients with BRCA1 heterozygocity.

FIG. 19. Sypro-ruby stained 5% SDS-PAGE of rodent DNA-EBC.

FIG. 20. Biotinylated oligo binds efficiently to avidin-bound magnetic beads. Supernatants from the binding reaction were electrophoresed. The addition of avidin beads (lane 1) reduced free oligo by 95% (compared to lane 2 with no beads).

FIG. 21. Representative DNA-EBC analysis of human tumor cell lines.

FIG. 22. Band A density correlates with SF2 for 15 independently derived human tumors.

### DESCRIPTION OF ILLUSTRATIVE EMBODIMENTS

### I. THE PRESENT INVENTION

As mentioned above, there are large numbers of people who are at risk of treatment-induced side effects from radiation therapy. Predicting the radiosensitivity of normal and tumor cells has great potential importance for radiation oncology. However, at present, there is no way to determine the radiosensitivity of tumor and normal tissue. Thus, there is a need in the art for a simple and cost effective method to assess the susceptibility of cells to DNA double-strand break (DNA damage), the most important cytotoxic lesion caused by radiation. The present invention overcomes the deficiencies in the art by developing DNA-end binding complex (EBC) pattern as an intermediate marker for radiosensitivity, using a band shift technique such as electrophoretic mobility shift assay (EMSA). The DNA-EBC pattern will be used to predict the surviving factor (SF2).

The present invention overcomes the drawbacks SF2 assays when applied to the clinic. First, SF2 requires that patient specimens be grown in culture. While this can be done rather easily from skin fibroblasts, the process still takes several weeks. As discussed above, such growth in culture is difficult to achieve for tumor specimens so that very low plating efficiency is the rule. This may well bias the results of any assay done on 0.01% of cells. Second, tissue culture is very time intensive for lab personnel, making it very expensive. Third, tissue culture is not a routine of clinical laboratories, further increasing the cost of implementation. Despite these problems, tumor cell SF2 seems to predict local control in human patients with some tumors. It is also clear that some tumors (e.g., lymphomas) are more radiosensitive (can be locally controlled with lower radiation doses) than others (e.g., glioblastomas, which are almost never locally controlled with any clinically tolerable radiation dose). However, even some lymphomas failed local treatment. Thus, being able to individualize dose to individual tumors is a desirable goal.

The basic underlying mechanism in DNA damage and repair is that when there is breakage in a chromatid, all the proteins involved in the DNA damage and repair should ideally bind to the two DNA ends to enable rejoining of the two broken DNA ends. In the event that the genes producing such proteins are mutated, the proteins are unable to bind to the DNA ends. The present embodiment of the invention makes use of this observation to determine the level of a cell's susceptibility to DNA damage by providing an end-labeled oligonucleotide to the proteinaceous extract of a cell sample. The protein, depending on whether it is mutated or non-mutated, may or may not bind to the DNA. This, in turn, will be reflected in the band shift obtained as a result of electrophoresis of the sample mixture. The band shifts obtained as a result of electrophoresis of the sample mixture are compared with a control from a normal cell. A change in band shift indicates an altered sensitivity of cells to DNA damage. Such band shift analyses are obtained using the EMSA technique as is described herein.

The present invention provides a method using blood or tissue extracts, which are mixed with radiolabeled DNA and electrophoresed. DNA-end binding by extract proteins causes band shifts which are predictive of radiosensitivity. This process takes about 3 days from the time the samples are obtained and can be done for about a thousand dollars, though this cost may be further reduced with automation. This method also provides an important tool for the prediction of the risk of occurrence of cancer. The band shift of the labeled oligonucleotide indicates altered susceptibility to DNA damage, which predicts altered risk of cancer in said individual.

It has been indicated that radiation is a powerful carcinogen for breast cancer (Bhatia et al., 1996). Furthermore, low dose radiation associated with mammographic screening also provides some risk for breast cancer (Mattson et al., 2000). If heterozygous mutations in tumor suppressor genes BRCA1 and BRCA2 lead to haploinsufficiency, this risk will be greater for BRCA carriers (Bucholz et al., 2000). Thus, the present invention also provides a method for predicting the presence of BRCA mutations in an individual, which are indicative of an increased risk of an individual to develop breast cancer. Currently, the estimates of the risks of having a germline mutation utilize only clinical parameters. Screening is often offered to individuals felt to have a 20% or greater probability of having a mutation, but the cost and availability of resources preclude complete BRCA1/BRCA2 gene sequencing for all interested individuals. The method of the present invention can determine which individuals can avoid the cost and labor associated with full gene sequencing. In the present invention, band shift of the labeled oligonucleotide indicates BRCA mutations, which predicts an increased risk of breast cancer in said individual.

As mentioned above, the present screening methods to estimate the risk of having a germline BRCA mutation are expensive and cumbersome and only offered to individuals who have a greater probability of having a mutation. The present invention provides a screening tool that enables one to determine an individual's risk of having a germline BRCA1 or BRCA2 mutation in an easy and cost-effective manner. Furthermore, the present invention also provides a method to identify new candidate genes that affect the risk of breast cancer development. The analysis of the components of the EMSA bands that correlate with radiosensitivity demonstrate the presence of proteins such as ku70, ku80, ATM, xrcc4, DNA ligase 4, xpA, p53, rad51, blm, and wm.

Further, the present invention provides a method for predicting the toxicity of a cancer therapy. Again, a protein extract from the cells of a cancer patient is mixed with a labeled oligonucleotide and subjected to electrophoresis. Band shifts are indicative of the toxicity to cancer therapy. Thus, a therapeutic index can be calculated for each individual. Since this method can also be used to determine the degree of sensitivity of the tumor to the DNA damaging cancer therapy (above), a balance can be struck as to what is the maximum DNA damage that can be caused to the tumor cell without creating a toxic effect on the normal cells of an individual.

These and other aspects of the invention are described below.

### II. DNA DAMAGE AND REPAIR IN CELLS

It has been observed that some individuals are more susceptible to DNA damage than others. There are various ways by which damage to DNA may occur such as radiation, chemicals, ultraviolet light, x-rays, gamma rays and random errors in DNA replication. The types of DNA damage include loss of a base, breaks in one of the DNA strands, addition of a methyl group to guanine, thymine dimer formation (linkage of two adjacent thymine bases on one of the DNA strands).

Cells respond in many ways to radiation. For example, the cell cycle is arrested, pro-apoptotic pathways can be activated, transcription and translation are altered, and the cells repair their DNA. The balance of these responses decides whether an irradiated cell will live or die. Many of these responses are initiated by PI-3 kinases, though many other signaling occurs. The diversity of responses suggests that there may be several types of complexes that form at DNA strand breaks, each with different kinase(s).

Repair of DNA double-strand breaks is dependent primarily on the non-homologous end joining (NHEJ) pathway, although cell cycle regulation and apoptosis pathways can affect SF2 as well. NHEJ requires DNA dependent protein kinase (DNA-PK), Ku70, Ku80 (DeFazio et al., 2002), xrcc4 and DNA ligase IV (Grawunder et al., 1998). A number of proteins have also been shown to assemble into “foci” at putative sites of DNA breaks. These include BLM, PML, hRAD51 and RP-A (Bischof et al., 2001), MRE1 1/NBS1/IAD50 (Zhao et al., 2000), DNA polymerase mu and gamma H2AX (Mahajan et al., 2002), RAD50, RAD51, gamma H2AX and BRCA1 (Paull et al., 2000). ATM plays an important role in both focus formation and DNA repair, as does DNA-PK, whose activity is essential for effective strand rejoining (Calsou et al., 1999). These observations demonstrate that DNA-repair complexes form at sites of DNA damage, that several types of repair complexes may form, and that these complexes are dependent on kinase activity of several different PI-3 kinases. It is not clear, however, how many different complexes form at DNA breaks or how many proteins may be present in each. While it is clear that repair is regulated by phosphorylation, the substrates for each kinase have not been completely identified. Unfortunately, the levels of DNA repair proteins have not been shown to be good predictors of SF2 since neither DNA-PK nor Ku protein levels correlate with SF2 in head and neck cancers (Bjork-Eriksson et al., 1999) nor did it correlate with proliferative potential (Ki-67, PCNA, LI, Tpot) or p53 expression (Bjork-Eriksson 1999a).

Large variations in DNA repair capacity have been demonstrated in otherwise normal human populations (Setlow, 1983; Berwick and Vineis, 2000; Mohrenweiser and Jones, 1998). These individual variations in DNA repair likely occur because of subtle polymorphisms which are common within DNA repair genes (Shen et al. 1998). Unfortunately, detecting such polymorphisms requires the identification of the culprit gene followed by gene sequencing, which are both time consuming and costly. Also, mutations that affect radiosensitivity may be quite subtle, affecting only sites of post-translational modification or the active sites of key enzymes.

Of particular relevance are cancer therapies that involve the use of DNA damaging methods. Radiation therapy includes the use of γ-rays, X-rays, and/or the directed delivery of radioisotopes to tumor endothelial cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 centigray for prolonged periods of time (3 to 4 weeks), to single doses of 2000 to 6000 centigray. Dosage ranges for radioisotopes vary widely, and depend on the half-life of the isotope, the strength and type of radiation emitted, and the uptake by the neoplastic cells.

Cancer therapies also include a variety of chemotherapies, for example, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate. Thus, predicting sensitivity to DNA damaging therapies in both normal and cancerous tissues permits one to design specific therapeutic regimens for patients such that they achieve the best results.

### III. SURVIVING FRACTION (SF2) IN PREDICTING RADIOSENSITIVITY

Prediction of normal and tumor cell radiosensitivity has potentially great clinical value. The present invention therefore provides a method of predicting normal and tumor cell radiosensitivity. The present invention contemplates the use of the surviving fraction of cells irradiated to a dose 2Gy (SF2), as a measure of cell radiosensitivity. SF2 has predictive value in that it represents cell survival after a fraction of the amount of radiation commonly delivered clinically.

SF2 is indicated to be predictive of radiosensitivity in both normal and tumor tissue. Detailed modeling of the potential benefits to both complication rate and tumor control (Mackay and Hendry, 1999) were in agreement with that of others (Burnet et al., 1994; Burnet et al., 1996; MacKay et al., 1998; Norman et al., 1988; Thames et al., 1992; Tucker et al., 1996; West and Hendry 1992). These results pointed out that both tumor control probability (TCP) and normal tissue complication probability (NTCP) can potentially be improved by individualizing treatment based on radiocurability. The benefits were dependent on the predictive power of the assay, but predicted benefits would be clinically meaningful even if the correlation between test results and TCP or NTCP is between 0.4 and 0.6. Even if the assay only stratifies patients and tumors into just three risk categories (low, medium, high), the potential gain in TCP was predicted to be between 22% and 33%. However, the lack of accurate SF2 determination, length of time, and cost, significantly limits the clinical applicability of the assay.

Normal cell radiosensitivity has been correlated with skin fibrosis after breast radiation (Johansen et al., 1996), particularly in patients that were treated with large doses per fraction (>2.7 Gy). Fibroblast SF2 correlated with the maximal toxicity grade for patients irradiated for breast cancer (Brock et al., 1995). In selected cases, patients with severe DNA repair deficits like AT can have their treatment tailored to their intrinsic radiosensitivity with good results. For example, an AT patient with medulloblastoma was treated with 0.6 Gy fractions to 15 Gy, based on the measured SF2 of his fibroblasts (Hart et al., 1987). This demonstrated that, at least in patients with severe repair deficits, treatment can be safely and effectively individualized. SF2 of normal fibroblasts can predict late toxicity from radiation in both head and neck and breast cancer. Tumor SF2 may also predict tumor metastatic potential (Lewis et al., 1996). SF2 has shown some predictive potential in some cancers.

A study by (Stausbol-Gron and Overgaard, 1999) compared SF2 of tumor cells and local-regional control. In 38 patients, tumors were biopsied, explants cultured in soft agar, and SF2 determined. No correlation was found between SF2 and locoregional control for these patients treated with radiation alone. Interestingly also, no correlation was found between tumor cell SF2 and fibroblast SF2, suggesting that these may be independent parameters. One caveat of this observation was that the plating efficiency was extremely low (only 1/38 was above 1%, and seven were about 0.01%), suggesting that SF2 may have been measured only on a small subset of tumor cells. In fact, five tumors had SF2 of 1.00, yet two of these patients had local control. Therefore, SF2 of small tumor subsets must not be representative of the entire tumor population.

A larger study (Bjork-Eriksson et al., 2000) of 84 curatively treated patients with head & neck cancer did demonstrate a significant correlation between tumor SF2 and local control (p=0.036), but not survival. Thus, SF2 was shown to be predictive of local control in head & neck cancer in the largest study with the longest follow-up. Similarly, investigators have found that cervical tumors with low SF2 are more likely to be locally controlled than similar tumors with high SF2 (Buffa et al., 2001; West et al., 1991). However, SF2 may not be predictive of local control for all tumors (for example, glioblastomas (Taghian et al., 1993)).

### IV. DNA DAMAGE AND BRCA GENE

BRCA1 and BRCA2 are tumor suppressor genes that play important roles in the processing of DNA damage. The BRCA genes play an important role in preserving genomic integrity. Both BRCA1 and BRCA2 co-localize with Rad51 (Scully et al., 1997; Sharan et al., 1997; Davies et al., 2001) in a protein complex that is important for the recognition, processing, and repair of double-strand DNA breaks. In addition, DNA damage promotes localization of BRCA1 on proliferating-cell nuclear antigen-positive replicating structures, implying involvement in a checkpoint response (Chen et al., 1998). Data suggest that homozygous mutations in either gene results in a radiosensitive phenotype, probably due to a dysfunction in double-strand break repair. For example, previous reports have demonstrated that cells with a homozygous BRCA1 mutation display diminished oxidative damage repair in the transcribed strands of DNA (Gowen et al., 1998) and have a diminished capacity for DNA end-rejoining (Zhong et al., 1999). BRCA1 associates with and is phosphorylated by ATM (Cortez et al., 1999). The ATM gene plays a critical role in double-strand break repair and mutations in ATM result in profound cellular radiosensitivity (Blocher et al., 1991; Baskaran et al., 1997). Both ATM and BRCA1 have been shown to be present in a large complex of repair proteins that may have a role in the sensing and processing of DNA damage (Wang et al., 2000).

The inventors have previously demonstrated that heterozygous germline mutations in BRCA1 and BRCA2 are associated with a statistically significant increase in cellular radiosensitivity, presumably through haploinsufficiency (Buchholz et al., 2001). In addition, they found that ATM-containing DNA end-binding complexes are reduced in cells from individuals with a heterozygous BRCA1 of BRCA2 mutation. Thus, the high lifetime risk for breast cancer development that is associated with germline mutations in BRCA1 or BRCA2 may be due to a dysfunction in the role these genes play in DNA repair pathways, with resulting genomic instability. Recently, an analysis of lymphocytes from individuals with germline heterozygous BRCA2 mutations found evidence of genomic instability in the constitutional karyotype, as evidence by rearrangements at 9p23-24 (Savelyeva et al., 2001).

There may be a number of molecular consequences if germline BRCA mutations are associated with haploinsufficiency. Evidence suggests that breast cancers arising in individuals with a germline BRCA mutation are associated with a loss of the wild-type allele within the tumor (Smith et al., 1992; Collins et al., 1995; Staff et al., 2000). A deficiency in double-strand break repair from haploinsufficiency would increase the probability of a loss of heterozygosity at the BRCA locus and increase the frequency of mutations in other tumor suppressor genes, such as p53.

### V. ELECTROPHORETIC MOBILITY SHIFT ASSAY (EMSA)

Electrophoretic mobility shift assay (EMSA) has been previously used to study protein complexes that bind at or near DNA double-strand breaks (Yu et al., 2001; Okayasu et al., 2000). EMSA is used for DNA-protein binding studies. Thus, the present invention employs the use of EMSA to develop DNA-EBC pattern as an intermediate marker for radiosensitivity.

In general, the EMSA assay is performed by incubating a purified protein, or a complex mixture of proteins (such as nuclear or cell extract preparations), with an end-labeled DNA fragment containing the putative protein binding site. The oligonucleotide ends function in the assay as double-strand DNA breaks. The reaction products are then analyzed on a non-denaturing polyacrylamide gel. The specificity of the DNA-binding protein for the putative binding site is established by competition experiments using DNA fragments or oligonucleotides containing a binding site for the protein of interest, or other unrelated DNA sequences. Labeled DNA that is bound by protein migrates more slowly than unbound DNA and appear as bands that are shifted relative to the bands from the unbound. This assay may also be conducted in a similar way using other labels, e.g., fluorescence labeling.

The assay of the present invention involves the use of a 144-base pair oligonucleotide which is radioactively end-labeled. The present invention contemplates that any length of an oligonucleotide may be used for this method and the oligonucleotide may be end-labeled with a dye, a fluorescence label, an enzyme or a chromophore. In a particular embodiment of the invention, a ³²P end-labeled oligonucleotide has been used. The present invention further contemplates the addition of the oligonucleotide to a protein extract. This extract may be a cellular or nuclear extract taken from a lymphocyte or a fibroblast cell originating from a sample of blood or tissue. A vast excess of unlabeled supercoiled plasmid is also added to the mixture to remove any DNA binding proteins not specific for binding to the ends of double-strand DNA. This mixture is then electrophoresed in a 5% acrylamide gel under non-denaturing conditions and subjected to autoradiography.

The bands obtained are subjected to densitometric scanning. The gel is placed into a cuvette containing an appropriate buffer such as TBE and subjected to scanning. Scanners are equipped with capability to output analog data directly to various computer data management systems, while converting the data to digital information. In addition to storing the scan as a digital reading, these programs can be used to integrate the area under the curves for each peak and thereby yield quantitative data. The relative intensity of each band may be calculated with respect to the control. The amount of each protein found within the major peaks can also be calculated.

In the present invention it has been shown that the EMSA of a proteinaceous extract of any human cell nuclei results in nine bands. The exact content of the bands has not been determined, but many proteins known to be important for DNA repair or sensing DNA damage have been found such as ku70, ku80, ATM, xrcc4, DNA ligase 4, xpA, p53, rad 51, blm and wrn. Because so many important DNA repair proteins are within these complexes, one can use the complex density, or possibly migration pattern, to predict susceptibility to DNA damage. Similarly, since DNA damage over many years is thought to induce cancers, defects in the repair of DNA damage, as measured by alterations in EMSA banding, might predict cancer risk. The present invention contemplates that the intensity of these bands may alter depending upon the sensitivity of cells to DNA damage. The absence of the ATM protein can be detected by profound changes in EMSA pattern. Data from rodent EMSA suggest that subtle differences in ku80 protein, which predict chromosome instability, can be detected by EMSA. The addition of antibodies to specific proteins to the EMSA reaction can also predict differences in chromosome instability.

### VI. PRIMARY FIBROBLAST CELLS AND LYMPHOCYTES

The present invention contemplates the use of fibroblast cells or lymphocytes to carry out the electrophoretic assay of the present invention. The cells may be obtained from a subject that has cancer or from a subject that does not have cancer. Fibroblasts can be grown in primary cultures without genetic modification for approximately 20 passages. This allows the assay to be performed without immortalizing the cells. It is clear that immortalization of cells artificially disrupts the cell cycle and may have an effect on the radiosensitivity assays (Aprelikova et al., 1999). This is particularly of concern for studies investigating BRCA1, since viral immortalization disrupts retinoblastoma (RB)-dependent cell cycle control and may also interfere with BRCA1 binding to RB (Aprelikova et al., 1999). Fibroblasts are appropriate cells for studying whether heterozygous mutations in a tumor suppressor gene correlate with cellular radiosensitivity. Furthermore, the results of fibroblast clonogenic survival assays also correlate with clinical radiosensitivity. The inventors have shown in previous studies that results of normal skin fibroblast clonogenic cell survival curves correlate with the probability of late normal tissue injury after ionizing radiation treatment, both in breast cancer patients and in a prospective study of patients with head and neck cancer (Brock et al., 1995; Geara et al., 1993). In addition, there have been other researchers who have all reported a significant correlation between in vitro fibroblast survival and the risk of normal tissue toxicity after radiation treatment of breast cancer (Burnet et al., 1992; Johansen et al., 1996; Hannan et al., 2001).

Lymphocyte may also be used to perform the assay of the present invention. The lymphocyte chromatid break assay has been used as a method for studying mutagen sensitivity (Hsu et al., 1989; Parshad et al., 1996; Helzlsouer et al., 1996; Patel et al., 1997; Scott et al., 1999). Lymphocytes have advantages over fibroblasts: they can be obtained by routine phlebotomy available ubiquitously, especially, in the USA. Also, they do not need to be cultured or purified as is needed for fibroblasts.

### VII. OLIGONUCLEOTIDES SYNTHESIS

The present invention contemplates the use of end-labeled oligonucleotides use in the present invention. The oligonucleotide may be of varying lengths.

Oligonucleotide synthesis is performed according to standard methods. See, for example, Itakura and Riggs (1980). Additionally, U.S. Pat. No. 4,704,362; U.S. Pat. No. 5,221,619; U.S. Pat. No. 5,583,013; each describe various methods of preparing synthetic structural genes.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Alternatively, oligonucleotides can be simply cut from plasmids (which can be grown by a variety of published techniques) and purified using commercially available kits.

Labeling Oligonucleotides. The present invention provides a label or a detection agent bound to the oligonucleotide. A label or a detection agent is defined as any moiety that may be detected using an assay. Non-limiting examples of labels or detection reagents that may be conjugated to oligonucleotides include radiolabels, dyes, haptens, fluorescent labels, phosphorescent molecules, chemiluminescent molecules, chromophores, luminescent molecules, photoaffinity molecules, colored particles. The examples that involve detection by color are generally understood to be colorimetric labels or detection reagents. Herein, “label” and “detection reagent” are used interchangeably.

Many appropriate imaging agents are known in the art. The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (TI), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for the method of the present invention, one might mention astatine²¹¹, ¹⁴carbon, ⁵¹chromium, ³⁶chlorine, ⁵⁷cobalt, ⁵⁸cobalt, ⁶⁷copper, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹ are also often preferred due to their low energy and suitability for long range detection. Radioactively labeled oligonucleotides of the present invention may be produced according to well-known methods in the art.

The fluorescent labels contemplated for use as labels include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Electrophoretic Mobility Shift Assay

Plasmid pUC18 was digested with both PvuII and EcoRI to generate a 144-bp probe (Getts and Stamato, 1994; Stevens et al., 2002). This probe was ³²P-labeled using the Klenow fragment of DNA polymerase I in the presence of [α-³²P]dATP (DuPont NEN), and the unincorporated nucleotide was removed by chromatography on Sephadex G-50 spin columns.

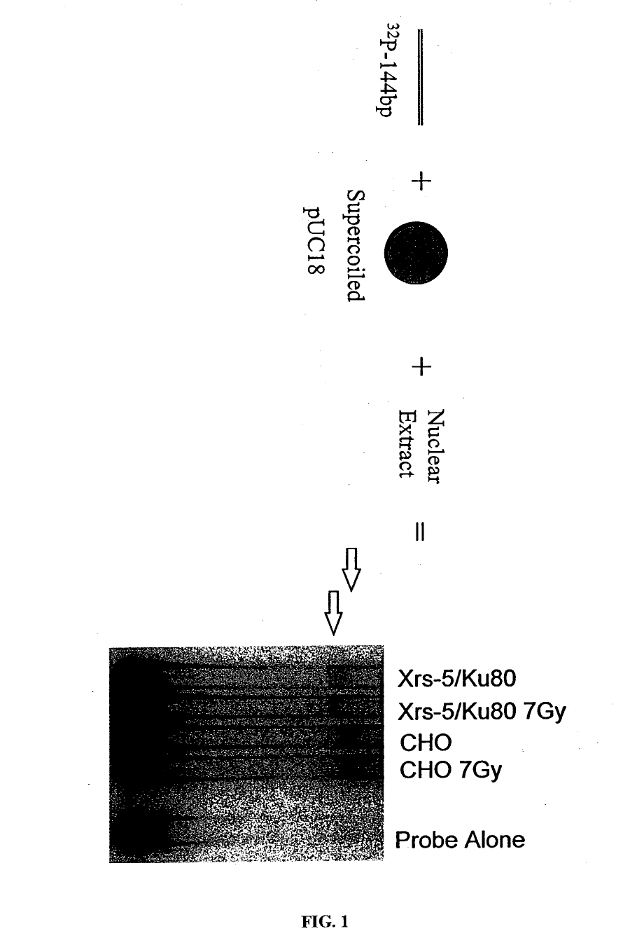

Nuclear extracts were made six hours after irradiation of intact cells or from unirradiated cells. This time was chosen because improved plasmid end joining activity has been detected 3-6 hours after irradiation (unpublished observations). Nuclear extracts (5 μg protein) were then incubated with 1.0 ng of labeled DNA probe for 15 minutes at room temperature in the presence of 410 ng of unlabeled, closed circular pUC18 plasmid (nonspecific DNA competitor) in a final volume of 20 μl in binding buffer (10 mM Tris-HCL, pH 8.0, 0.1 mM EDTA, 150 mM NaCl, 1 mM DTT, 1 mM PMSF, and 10% (v/v) glycerol). A vast excess of unlabeled supercoiled plasmid was added to the reaction to remove proteins, such as histones, that do not bind specifically to DNA ends. The mixtures were electrophoresed on a 5% polyacrylamide gel at 20-25 mA in TBE buffer (45 mM Tris-HCL, pH 8.0, 45 mM boric acid, 1 mM EDTA) under non-denaturing conditions and were subsequently dried and subjected to autoradiography. The alterations in electrophoretic mobility of the radiolabeled oligonucleotide were then used to identify end-binding protein complexes (FIG. 1).

### EXAMPLE 2

### Study of EMSA Bindine Patterns

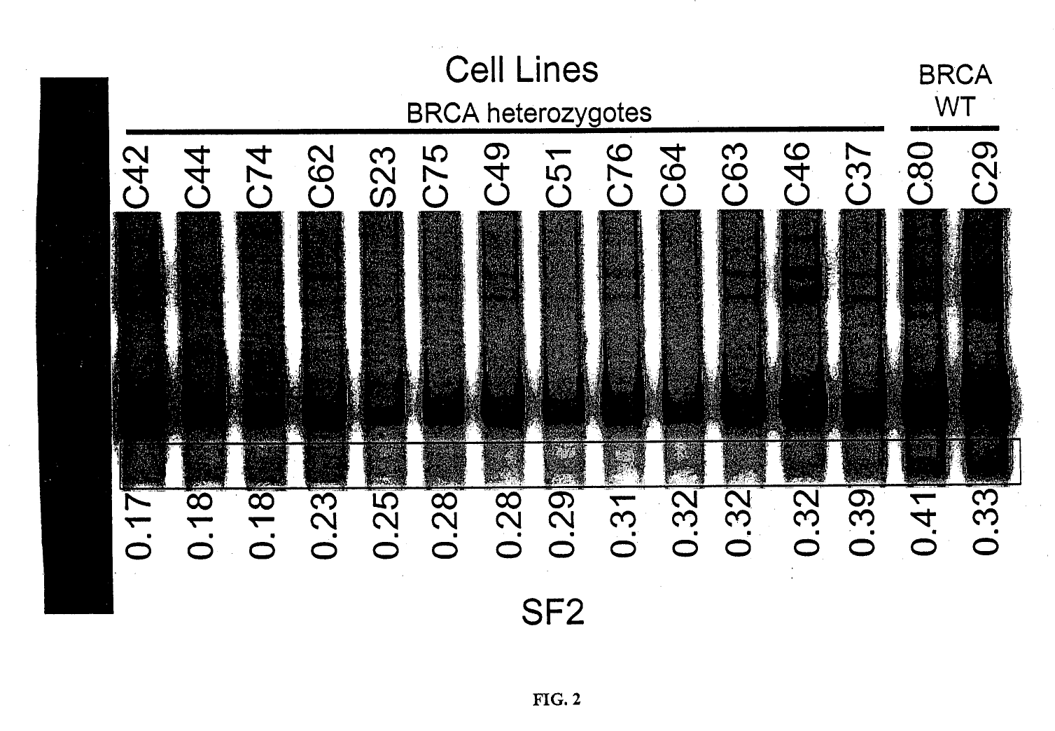

EMSA binding patterns have been compared for cell lines with a variety of radiosensitivities. Ten separate bands were identified in normal controls and 10 primary fibroblast lines from patients heterozygous for germline BRCA mutations were then obtained that were previously shown to have a range of radiosensitivities. The Surviving Fraction (SF2) ranged from 0.19-0.39 (FIG. 2). Fibroblast lines obtained from cancer patients without BRCA mutations, and one fibroblast line from a patient related to a BRCA heterozygote but with sequence-normal BRCA, were used as controls. The SF2 ranged from 0.39 to 0.44 (data not shown).

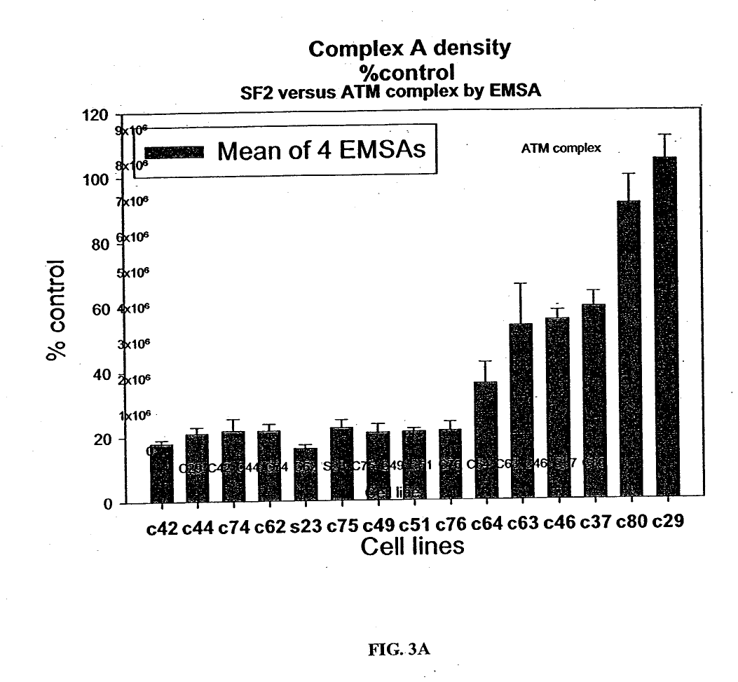

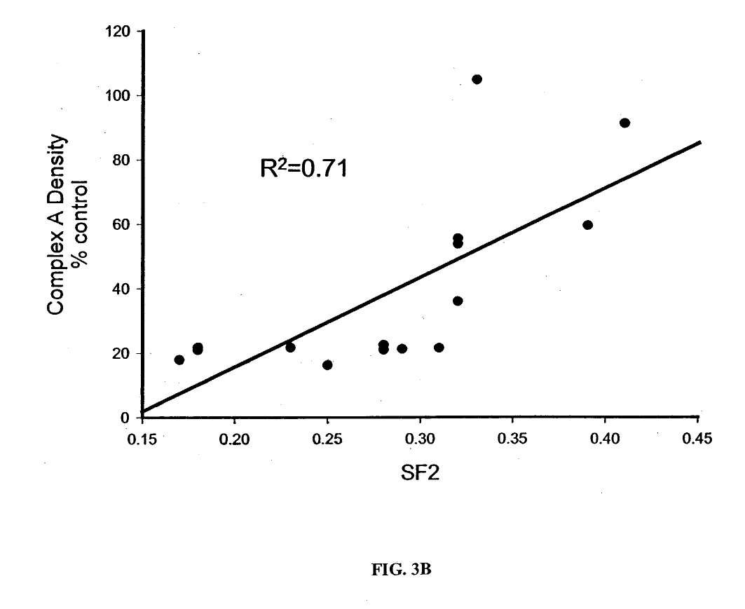

EMSA analysis of the BRCA heterozygotes demonstrated reduced intensity in 6 of the 10 EMSA bands (FIG. 3A). There was a good correlation between the intensity of these 6 bands and SF2 (R²=0.71) (FIG. 3B). EMSA analysis was also performed on 10 other fibroblast lines from cancer patients without BRCA mutation but with a variety of SF2 values. Again, a good correlation was seen (R²=0.75) (data not shown). Thus, EMSA analysis can predict radiosensitivity of primary fibroblasts from cancer patients. EMSA analysis has also been performed on two leukemia cell lines (surrogates for peripheral blood leukocytes) and the EMSA banding pattern was identical to that of normal control fibroblasts.

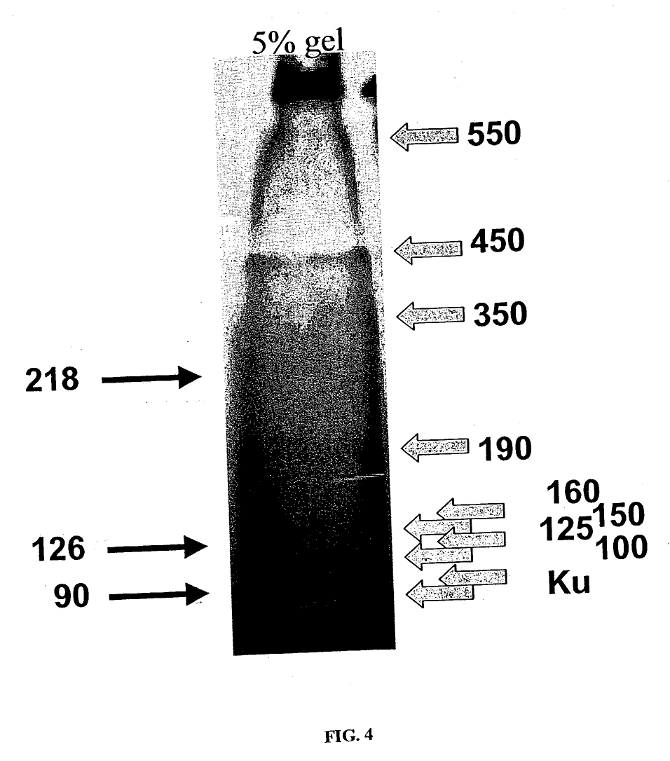

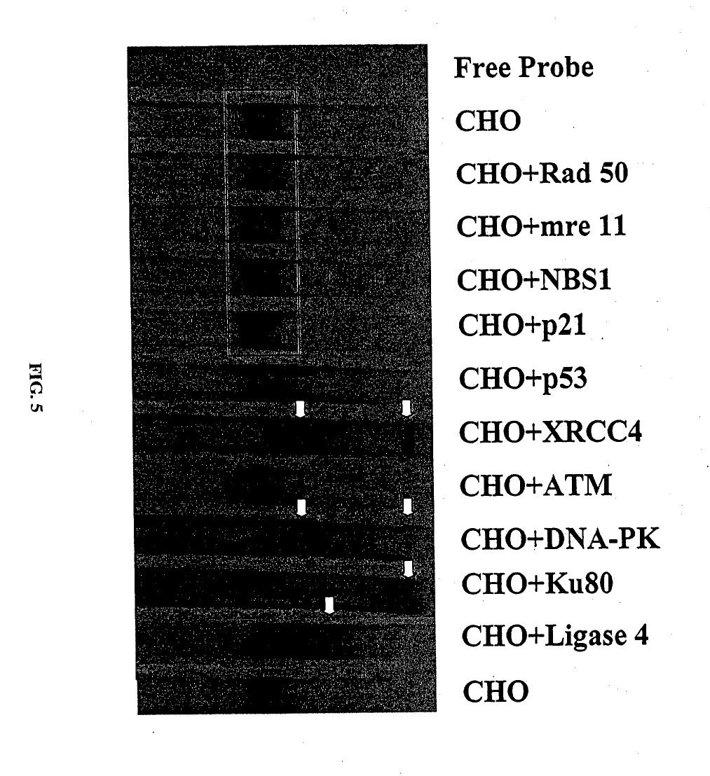

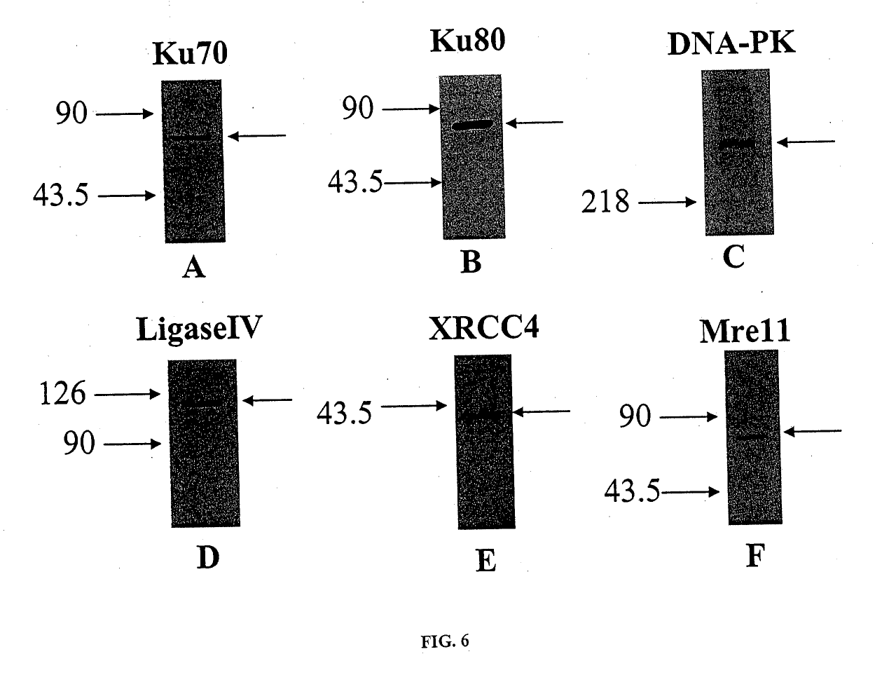

DNA end binding complexes were eluted. The number and molecular weights of ³⁵S-methionine-labeled proteins in the purified EMSA complexes were determined using SDS-PAGE and autoradiography (FIG. 4). Individual proteins within the EMSA complex were identified by supershift analyses, which are performed by adding specific antibodies to candidate proteins with appropriate molecular weights (FIG. 5). The following proteins were identified from the analyses: Rad50, mre 11, NBS1, p21, p53, XRCC4, ATM, DNA-PK, Ku80, Ligase 4. Western blot analysis of purified EMSA complexes was used to confirm the presence of these individual proteins (FIG. 6)

### EXAMPLE 3

### BRCA Germline Mutations in Human Cells Affect Molecular Complexes that Assemble at the Sites of Double-Strand Breaks

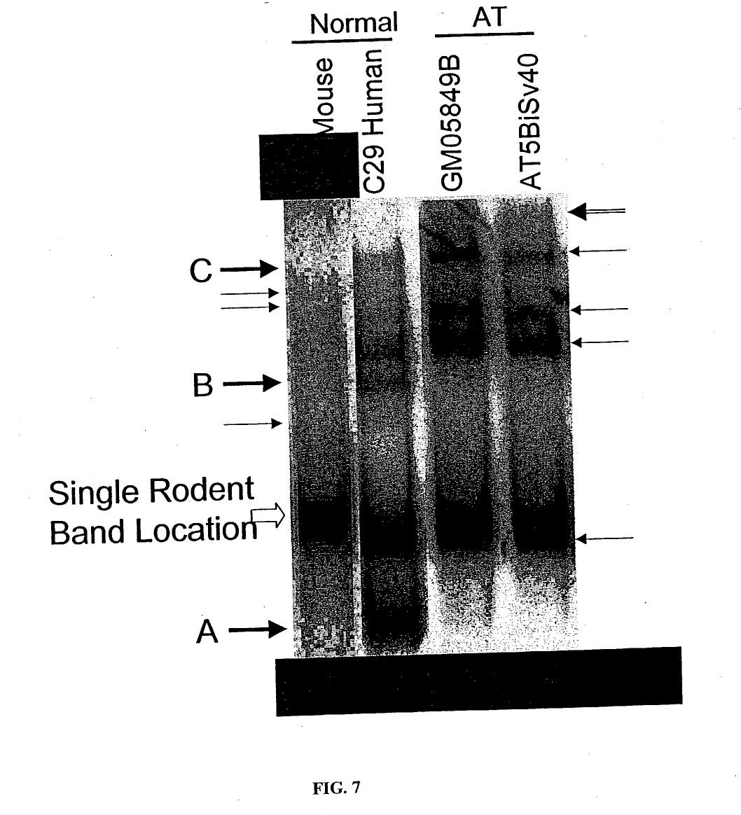

Tests were conducted to determine how BRCA germline mutations in human cells might affect molecular complexes that assemble at the sites of double-strand breaks. Initial observations found that EMSA banding pattern from normal human fibroblast nuclear extracts were dramatically different than those from rodent fibroblast nuclear extracts (FIG. 7, lane 1 and lane 2). Using human fibroblasts, 10 molecular complexes in normal fibroblasts were identified that bind to double-strand breaks (FIG. 7, lane 2). In fibroblasts with homozygous ATM mutations (FIG. 7, lanes 3 and 4), three major (A, B, and C) and three minor (small arrows at left) complexes were missing. In addition, one unique band was seen only in ATM cells (FIG. 7, bolded arrow at the right of the figure).

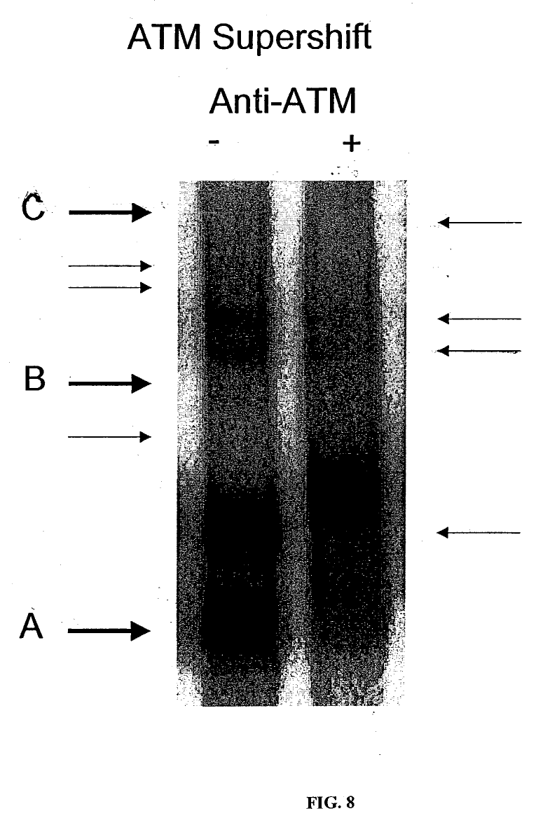

Preliminary characterization of the EMSA band components in normal cells was performed using gel supershift techniques. EMSA supershift with anti-ATM antibodies altered the migration of the major bands, demonstrating that ATM was present in these complexes (FIG. 8). Additional supershift data confirmed the presence of Ku70 and Ku80 in many complexes (data not shown). These data together suggest that the ATM protein was present in several of these complexes and that supershift analysis can be a good screening tool for putative EMSA components.

Because ATM has been shown to interact with BRCA proteins, it was determined whether the radiosensitivity seen in BRCA heterozygotes might be reflected by a change in EMSA banding patterns. Initial studies of fibroblasts from carriers of heterozygous BRCA mutations revealed that the ATM-containing complexes were diminished compared to fibroblasts from individuals with normal BRCA genes (FIGS. 2 and 3A, complexes A, B, and C).

In the one matched BRCA1 heterozygote with a kindred normal control, the differences in band intensities were dramatic (C75 (BRCA1 heterozygote)) vs. C80 (matched normal kindred of the donor of C75). Specifically, the intensity of complex A in C75 was only 30% that of C80. Complex A density was then correlated for all cell lines with the clonogenic survival results (SF2) (FIG. 2, white box at arrow A). Complex A was selected for this analysis rather than B or C, because it had the greatest intensity and was more separated from adjacent complexes. FIG. 3A shows the mean density of band A from four replicated experiments and the corresponding SF2 values. FIG. 3B shows a plot of this relationship. As shown, there was a strong correlation between complex A density and SF2, with a R² value of 0.71. Differences in SF2 between cell lines from BRCA carriers may be indicative of specific mutations within BRCA loci. These data suggest that BRCA haploinsufficiency can alter the amount of a DNA end-binding complex that contains ATM, and that the level of this complex correlates with radiosensitivity.

### EXAMPLE 4

### DNA-EBC Techniques

DNA-EBC Assay

The DNA-EBC assay is identical to that published for the analysis of DNA-EBCs from rodent cells (Stevens et al., 1999). Briefly, equal amounts of nuclear extract from each cell line of interest are mixed with a radiolabeled 144 bp oligonucleotide (which has ends, similar to DNA double strand breaks), and a vast excess of unlabeled supercoiled plasmid (which will bind proteins like histones that do not bind specifically at DNA ends). Electrophoresis under non-denaturing conditions allows the separation on unbound oligonucleotides from those with DNA-EBCs, which can be resolved by autoradiography (of the radiolabeled oligo). Antibodies can be added to the DNA-EBC reaction prior to loading to generate supershifts. These antibodies can be used to identify proteins present within a DNA-EBC band.

Affinity purification of DNA-EBCs

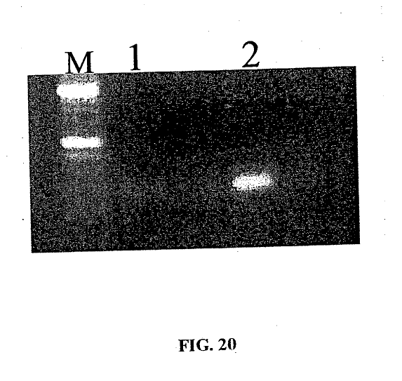

An affinity purification technique was developed to isolate DNA-EBCs in quantities sufficient for mass spectrometry. In this assay, one of the PCR™ primers used to generated the 144 bp oligonucleotide was biotinylated. This resulted in an oligo that was labeled at only one end. These biotinylated oligonucleotides can then be bound to avidin-linked magnetic beads, with one end always unbound. As can be seen in FIG. 20, the amount of oligo can be titrated so that the beads are maximally loaded. The oligo-loaded beads (which each have one free end because only one end of the oligo is biotinylated) can then be mixed with nuclear extract and a vast excess of supercoiled plasmid (without ends) to remove non-end-binding proteins. The beads are then washed, boiled to remove proteins (high salt washes were ineffective at removing proteins), and the proteins used for SDS-PAGE followed either for mass spectrometry or western analysis (e.g., with anti-phosphotyrosine antibodies, etc.).

### EXAMPLE 5

### DNA-EBC Pattern Predicts SF2

It was noticed that the pattern of DNA-EBCs varied between cell lines (Nimura et al., 2002). It was hypothesized that, since DNA double-strand breaks are the most relevant lesions for cell survival, the pattern of protein binding to DNA double-strand breaks might predict DNA repair capacity (and so radiosensitivity). To test this, DNA-EBC analysis was performed using nuclear extracts from cells with a variety of radiosensitivities. Primary human fibroblast cell lines were derived, over the years, from several different protocols. One protocol involved the acquisition of primary fibroblasts from patients with abnormally severe radiation reactions. Another protocol involved the generation of primary fibroblast cultures from patients irrespective of their radiosensitivity (Geara et al., 1993). A third protocol involved the generation of primary fibroblast cultures from patients heterozygous for BRCA mutations (Buchholz et al., 2002). It was noted that there were at least 10 bands present in DNA-EBC gels from normal primary human fibroblasts, but the relative abundance of each band was rather variable. However, the relative intensity of the band labeled “band A” markedly decreased as SF2 decreased. A representative DNA-EBC analysis is shown in FIG. 2.

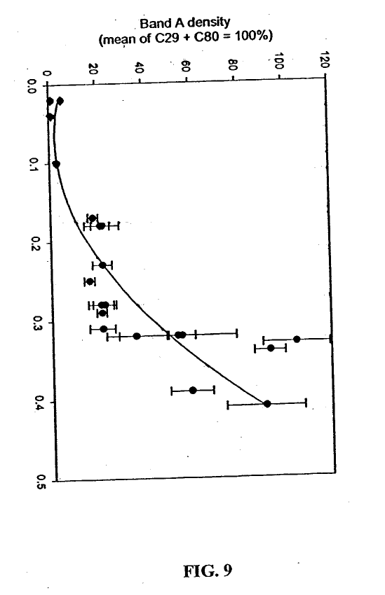

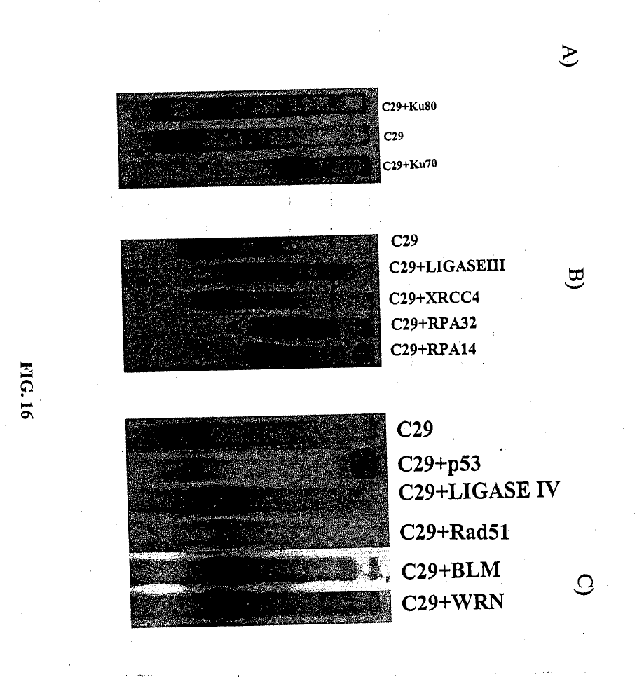

It was noticed that the most rapidly migrating band (labeled “band A”) varied in intensity quite markedly in the different primary cell lines. Several other bands also seemed to decrease with decreasing SF2, in particular those shown by arrows to the left of FIG. 2. Band A was studied for several reasons. First, the other bands were either very faint, or migrated very close to bands which did not have SF2-dependent density. Second, the analysis of band components is easier for more rapidly migrating bands (see supershift assays below, e.g., FIG. 16). When band A density (relative to that of the C29 control done on each gel) is plotted vs SF2 (FIG. 9), a quadratic regression line fits the data very well (correlation coefficient 0.82). Note that DNA-EBC analysis would also separate these cells quite well into groups (SF250.1,0.1<SF2<0.3, and SF2>0.3). Thus, band A density predicts SF2 in these 20 cell lines. These cell lines are described in Table 1. Note that these represent cell lines with marked radiosensitivity (ATM mutants and BRCA1 homozygous mutant), intermediate radiosensitivity (BRCA heterozygotes and some cell lines from patients with marked radiation reactions), and high-normal radiosensitivity (two unrelated normal lines). The observation that band density rather than band migration speed (or perhaps broadness of the band) predicts SF2 has mechanistic implications.

TABLE 1
Description of primary cell lines for which band A
density and SF2 have been studied

| Cell Line | Source | Known Mutations? |
|---|---|---|
| C42 | Protocol 98-212 | Cancer syndrome |
| C44 | Protocol 98-212 | BRCA1 |
| C74 | Protocol 98-212 | Grade 3 acute reaction, breast cancer |
| C62 | Protocol 98-212 | BRCA1 heterozygote |
| S23 | Protocol 98-212 | Familial breast cancer |
| C75 | Protocol 98-212 | BRCA1 heterozygote |
| C49 | Protocol 98-212 | BRCA2 heterozygote |
| C51 | Protocol 98-212 | BRCA1 heterozygote |
| C76 | Protocol 98-212 | BRCA1 heterozygote |
| C63 | Protocol 98-212 | BRCA1 heterozygote |
| C46 | Protocol 98-212 | BRCA1 heterozygote |
| C37 | Protocol 98-212 | Cancer syndrome |
| C80 | Protocol 98-212 | BRCA wt, daughter of c75, normal |
| C29 | Protocol 98-212 | Normal patient |
| C19 | Protocol 98-212 | Passage dependent SF2 |
| HCC1937 | ATCC | BRCA-both alleles |
| GM03396A | Coriell | AT |
| GM03395C | Coriell | AT |
| GM05849B | Coriell | AT |
| AT5BISV40 | Coriell | AT |

### EXAMPLE 6

### Development of the DNA-EBC Assay for Use in Lymphocytes

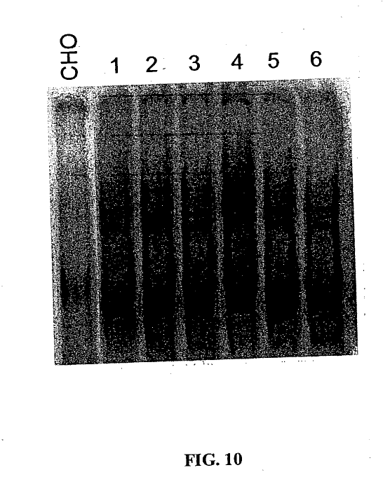

All of the above studies used nuclear extracts from primary fibroblasts. If such a test were to be developed for use in the clinic, it would be better if DNA-EBC analysis could be done on more easily available samples than fibroblasts, such as peripheral blood lymphocytes. In developing a lymphocyte-based DNA-EBC analysis, the DNA-EBC pattern in two primary fibroblast lines (FIG. 10, lanes 1-2) was compared with the DNA-EBC patterns from four peripheral blood lymphocytes samples from unrelated individuals (FIG. 10, lanes 3-6). The DNA-EBCs from fibroblasts and lymphocytes are indistinguishable. Fibroblasts and lymphocytes from one individual who is heterozygous for an inactivating ATM mutation have been obtained.

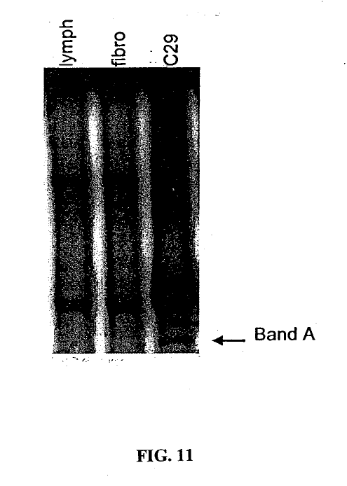

Buffy coat cells were derived from heparinized blood using commercially available (Sigma, St. Louis, Mo.) density gradients. After washing the cells, nuclear extracts were prepared. Using this technique, the DNA-EBC pattern was found to be similar for lymphocytes and fibroblasts derived from this patient (FIG. 11). Relatively less band A was found in both lymphocytes and fibroblasts than C29 normal control. SF2 is not yet available for these fibroblasts. This provides support for the principal that DNA-EBC analysis of peripheral blood lymphocytes should be similar to matched fibroblasts. Since previous studies have demonstrated that fibroblast SF2 is a very good predictor of long term toxicity from radiotherapy, DNA-EBC analysis of patient lymphocytes as a predictor of toxicity will be tested.

### EXAMPLE 7

### Tumor Cells and Radiosensitizers

The question of whether, the DNA-EBC pattern can also be used to predict SF2 of tumor cells was addressed given that DNA-EBC pattern can predict SF2 of primary cells. Several lines of evidence suggested that DNA-EBC pattern can predict the radiosensitizing effects of a variety of novel radiosensitizers on tumor cell lines. This is perhaps as important as determining the SF2 in untreated tumor cells.

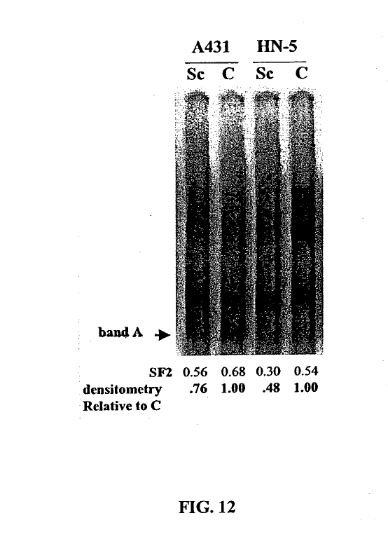

COX-2 inhibition has been shown to radiosensitize a variety of tumor cell lines (Kishi et al., 2000). Tumor cell lines (A431 and HN-5) that had been treated with SC236 (a COX-2 inhibitor) were obtained. As can be seen in FIG. 12, band A is reduced in the cell lines treated with radiosensitizing doses of relative to controls. Controls were either untreated or SC236 treated, but unirradiated cells. Note that the relative change in SF2 is very similar to the relative change in band A shown at the bottom of the figure. Interestingly, in HN-5 cells there are also changes in some of the upper bands. This may be indicative of the mechanism of radiosensitization by SC236. Once the components of each band are identified, it will be possible to choose proteins present in these bands, but not in other bands, to study as potential targets for COX-2 mediated radiosensitization.

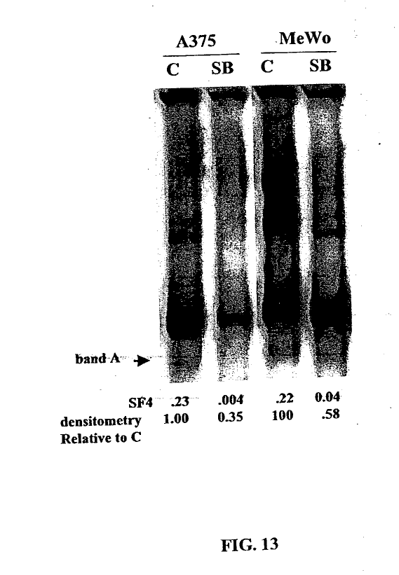

Similarly, the histone deacetylase inhibitor sodium butyrate has been shown to be a radiation sensitizer. Cells were obtained that had been treated with radiosensitizing doses of sodium butyrate in vitro. FIG. 13 demonstrates that the radiosensitization parallels the reduction in band A density. For this compound, there may also be a subtle shift in the migration rate of the lowest two bands (SB runs slightly faster), but not the higher bands. A more detailed drug-DNA-EBC relationship will be determined. The altered mobility rate may reflect the mechanism of radiosensitization. One potential target for SB would to increase acetylation of histone gamma H2AX. Data from nuclear focus forming experiments (Mahajan et al., 2002) would certainly place gamma H2AX near sites of DNA strand breaks.

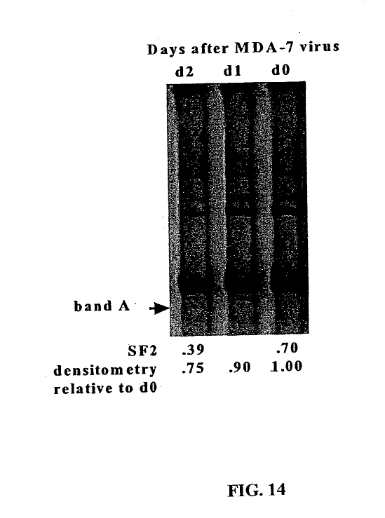

One other radiation sensitizer is MDA-7, a tumor suppressor gene that has been recently identified (Pataer et al., 2002). Gene replacement therapy with an adenovirus expressing the full length MDA-7 gene is both pro-apoptotic and radiosensitizing. A549 human lung carcinoma cells were obtained that had been treated with radiosensitizing doses of MDA-7 gene therapy in vitro, and the DNA-EBC pattern in MDA-7 treated cells and untreated controls determined. FIG. 14 demonstrates that radiosensitization MDA-7 gene therapy can be predicted by DNA-EBC pattern.

These observations represent single experiments. However in all examples, band A density parallels radiosensitivity. This provides evidence that changes in DNA-EBC can predict radiosensitization.

### EXAMPLE 8

### Developing the DNA-EBC Assay to Predict SF2 of Cells in Tumors

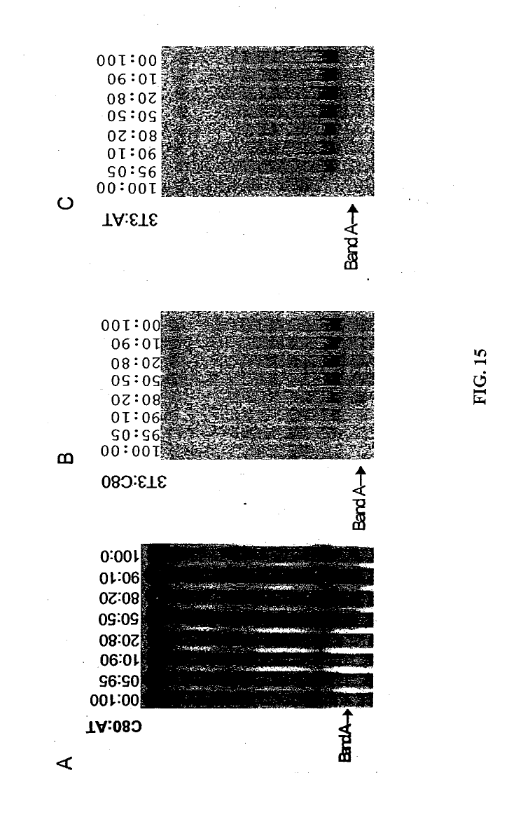

Determining DNA-EBC patterns in tumors (not cell lines) is somewhat complex. This is because there will be some normal cell contamination (rodent cells if the study is done in experimental animals, or normal human cells if the study is done on a human biopsy specimen). Also, there can be contamination with necrotic tissue or fibrous tissue, both of which could cause artifacts because the assay is performed using standard amounts of protein (thus protein from other sources would dilute the percentage of nuclear proteins in the assay). To overcome these problems, tissue micro-dissection is required. However, to determine the appropriate micro-dissection technique, it is important to know how contaminating normal cells might affect the DNA-EBC pattern. Therefore, nuclear extracts from normal human fibroblast (SF2=0.4) were mixed with nuclear extracts from cells with homozygous mutation in ATM (FIG. 15, panel A). Densitometry demonstrates that the radiosensitive phenotype could be well predicted by DNA-EBC analysis when less than 20% contamination occurs with cells with a higher DNA-EBC density. Nuclear extracts from normal cells were also mixed with extracts from NIH/3T3 mouse cells. FIG. 15 (panel B), demonstrates that the DNA-density/pattern is very stable until there is more than 20% contamination with rodent proteins. Perhaps most interesting is the result of mixing rodent extracts with those from AT cells (FIG. 15, panel C). Rodent extracts have no effect on band A (it is still undetectable) at any mixing ratio. Since rodent ATM and human ATM are very similar, this suggests that AT activity is required for band A assembly. ATM does not simply play a structural role. These mixing studies will be modify by adding ATP to the DNA-EBC reaction to determine if band A can form when AT activity is present. These observations demonstrate that relatively simple micro-dissection techniques can be applied, in contrast to those required for RT-PCR which is sensitive to even tiny amounts of contamination. The isolation of nuclear proteins from tissue requires additional optimization.

This technique can be used to guide therapy, since the radiosensitizing effects of biologic therapies can be predicted in individual patients, or class solutions developed once the range of individual variation in effects are known. The timing of radiation with respect to drug delivery may be optimized by measuring the intratumoral effects.

### EXAMPLE 9

### Molecular Characterization of DNA-EBCs

In order to understand the mechanism by which DNA-EBC pattern predicts radiosensitivity, it is essential to first determine the components of band A. Significant progress has been made in determining the components of band A.

Since AT cells are particularly radiosensitive and since the ATM protein was thought to bind at sites of DNA breaks, it was hypothesized that ATM might be an important component of band A. To test this, the DNA-EBC pattern of two cell lines derived from patients with ataxia telangiectasia was determined. As can be seen in FIG. 7, band A is essentially undetectable (lanes 2 and 3) in AT cells. In fact, three major (A, B, and C) and 3 minor (small arrows left) bands were missing in both ATM mutants compared with a normal control (FIG. 7, lane 1). One unique band was observed in the ATM cells (double arrow). Also, the relative intensity of bands is different in AT cells than controls, with some bands relatively more intense while others are less intense. This suggests that mutations in ATM cause widespread changes in the complexes that form at DNA double-strand breaks, even those that do not contain detectable ATM protein. To determine whether ATM is a component of any of these missing bands or simply affecting the DNA-EBC pattern by another mechanism, a supershift analysis was performed. When anti-ATM antibody was added to the DNA-EBC reaction from normal cells (FIG. 8), each of the six bands missing from the ATM mutant cells was supershifted. This was most apparent in the most intense bands (A and B) which were completely supershifted. Interestingly, the intense band above “A” was also supershifted. The relative intensity of this band was reduced in AT cells compared with control in FIG. 7, perhaps because ATM is a small component of this band. The effects cannot be explained by the presence of two independent complexes at this location, because the band in FIG. 8 is completely supershifted. These data demonstrate that several bands (including band A) contain ATM. Several other bands probably do not contain ATM because they are not reduced in AT cells nor supershifted by anti-ATM antibody, however rigorous characterization of each band is required to confirm this.

It has previously been suggested that BRCA1 heterozygocity results in a state of haploinsufficiency wherein BRCA heterozygotes are more radiosensitive than normal cells (Buchholz et al., 2002). Because this mechanism of radiosensitization is likely due the a single cause (BRCA1 mutations), it was reasoned that an analysis of the DNA-EBCs from these unrelated patients could provide mechanistic insights into both reasons for radiosensitivity, and the predictive power of DNA-EBC analysis (these cell lines were included in the data shown in FIGS. 2 and 9). Therefore, the band A components in these BRCA1 heterozygotes were analyzed.

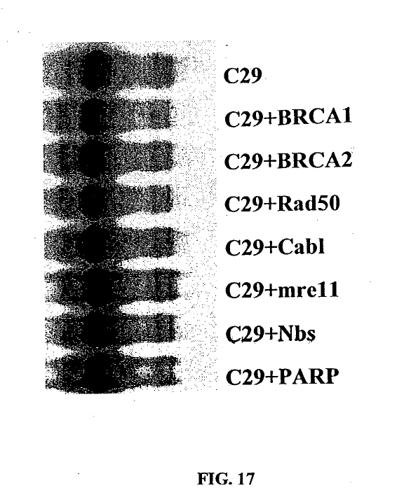

In determining the protein components of band A, it was hypothesized that the components of the DNA-EBCs were likely to be either DNA-repair proteins, or proteins known to interact with ATM. To test this, antibodies to such proteins were added to the DNA-EBC assay of normal (C29) cells, and the resulting supershifts determined. FIG. 16, panels A-C, demonstrate the presence of Ku70, DNA ligase III, DNA ligase IV, XRCC4, RPA32, RPA14, p53, Rad51, BLM, and WRN within band A. FIG. 17 demonstrates that Ku80, BRCA1, BRCA2, Rad50, c-abl, NBS1, and PARP were not shown to be present by supershift analysis. This does not rule out the presence of these proteins because the relevant epitopes could be blocked by other proteins. Also, band A never partially supershifted, suggesting that band A may be a single protein complex. Unfortunately, the complicated supershifting patterns usually preclude a comprehensive analysis of the more slowly migrating bands because of band overlap. However, Ku70, Ku80, and RPA seem to be present in most complexes because antibodies to these proteins supershift to the uppermost regions of the gels.

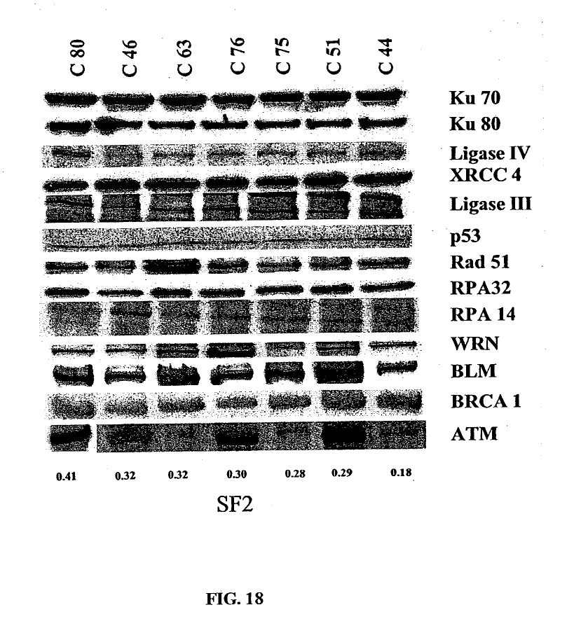

One simple explanation for a reduction in band A density would be that a BRCA mutation reduces the level of key band A components. As these components are reduced, the band A density might decrease as well. To test this, Western analysis (FIG. 18) was performed on nuclear extracts from the control (C80) and each of the BRCA1 heterozygote cell lines (C46, C63, C76, C75, C51, C44) with antibodies to each of the proteins identified in FIG. 8 to be present within band A (FIG. 17). There was no correlation between SF2 and the levels of any protein found thus far in band A (ATM, Ku70, DNA-PK Ligase III, Rpa32, Rpa14, DNA ligase IV XRCC4, WRN, BLM, RAD51 or p53). Densitometry was performed on each band, corrected for β-actin loading, and the results plotted vs. SF2. The r² values ranged from <0.001 to 0.49, in contrast with the r² value for band A density which was 0.85. Importantly, there was no correlation with BRCA1 or ATM protein levels and band A (or SF2).

Current data suggests that band A represents a single complex. First, band A is not partially supershifted by any antibody, as might be expected if many different complexes of similar molecular weight migrated together by chance. Second, band A is not supershifted by anti-Ku80 antibodies, while all 9 other bands are supershifted. This is unusual, and suggests either a complete Ku80 epitope blockade (since polyclonal antibodies were used in the supershift analysis), or the absence of Ku80 from band A. Third, if many complexes were present, it would be expected that removal of one protein (such as ATM) would not completely eliminate band A. Also, several of the proteins found in band A have been found to localize in nuclear “foci” that form after radiation (Bischof et al. 2001). In particular, BLM, PML, hRAD51 and RPA (three of which are in band A) have been found to co-localize at sites of putative DNA strand breaks. Other proteins that putatively bind to DNA double-strand breaks include the ATM-dependent heterotrimer MRE11/NBS/RAD50, which was not found in band A. This suggests that many types of DNA-EBCs may occur in vivo, and may be represented by the different DNA-EBCs found by assay. Interestingly, data from other groups suggest that very large DNA repair complexes can form at DNA strand breaks such as BASC (Wang et al. 2000), although band A is not likely to be BASC for several reasons. These lines of reasoning suggest that band A is distinct from the other bands, and most likely a single complex. However, it might be expected that DNA repair complexes would be sticky, so it is certainly possible that these bands represent groups of complexes. But, even if band A is not a single complex, it is clear that the density of this band strongly correlates with SF2, and so is potentially of great value irrespective of the true nature of the band components. Also, determining the components of each of the DNA-EBCs may direct further study of in vivo DNA repair complexes.

The observation that band density rather than band migration speed (or perhaps broadness of the band) predicts SF2, has mechanistic implications. It suggests that assembly of the entire complex is somehow closely regulated. It does not seem to be partially assembled (moving more rapidly); it is (with the possible exception of histone deacetylase inhibition) an all-or-nothing affair. The mechanism by which this occurs can be dependent on the protein levels of key proteins as in the case of ATM. However in other cases, such as BRCA1 mutations and possibly histone deacetylase inhibition, DNA-EBC pattern is independent of the levels of any particular component (at least those known so far). The initial hypothesis was that the band density might fall with the level of key protein components. It was initially thought that the most likely affected components would be the Ku proteins because they have been shown to nucleate the binding of DNA-PK and other DNA repair proteins to the site of DNA breaks (Dynan and Yoo, 1998). However, intranuclear Ku70 levels did not change in an SF2-dependent way. While it is possible that radiosensitivity will correlate with the level of some yet-to-be-identified protein component, there is certainly no evidence from studies conducted that BRCA haploinsufficiency systematically alters any protein levels. These observations suggest that assembly (or possibly complex stability) may be regulated by post-translational modification of key component(s), perhaps by ATM-dependent phosphorylation, although other mechanisms are certainly possible.

These observations make DNA-EBC analysis fundamentally different from other approaches for estimating DNA repair capacity, for example, a proteomic approach to radiosensitivity prediction. DNA-EBC formation requires that all of the relevant proteins be properly modified and in a conformation that allows complex formation. This type of functional analysis cannot be easily done by other techniques. Perhaps the closest technique would be the determination of DNA double-strand break repair capacity (which requires cultured cells—a time consuming and costly process) because it also requires that repair proteins work together. The technique used in the present invention has the advantage that, once the complex components have been identified, the mechanism(s) of repair deficits/enhancements may be classify.

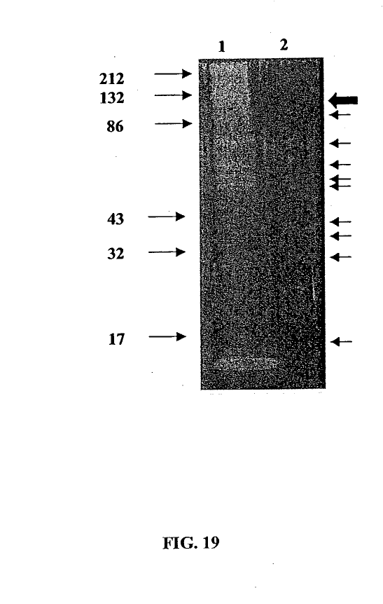

To identify all of the proteins in all of the DNA-EBCs a method for purifying DNA-EBCs in quantity sufficient for mass spectrometry was developed. The initial approach was to study the content of the much simpler rodent DNA-EBC (which is a single band as shown in FIG. 10). From the equivalent of about 1000 DNA-EBC reactions worth of nuclear protein the sypro-ruby stained proteins shown in FIG. 19 (lane 1) were isolated. In contrast, the use of beads without oligo demonstrated no nonspecific protein binding (lane 2), thus all of the protein seen in lane 1 were bound to the oligo. The reaction can be economically scaled up to the equivalent of 50,000 DNA-EBC reactions, which will allow the identification of rare proteins. This analysis demonstrates 10 bands with molecular weights ranging from 120 kd to about 12 kd. Previously, a very labor-intensive approach using nuclear extracts from ³⁵S-methionine labeled cells demonstrated a similar number of proteins in this molecular weight range, but the bands were quite difficult to visualize. Analysis by mass spectrometry has thus far identified poly ADP(ribose) polymerase (dark right arrow) with an apparent molecular weight of about 120 kD. This is similar to the published molecular weight of 113 kD. ³⁵S analysis of the rodent DNA-EBC demonstrated a protein at about 120 kd that had not yet been identified, which corresponds to the PARP identified by mass spectrometry.

Thus, the present invention provides a very powerful technique for the functional analysis of intranuclear DNA-end binding proteins. Once all proteins that bind to DNA ends have been identified, their DNA-EBC band localization can be determined by western (unfortunately the purification technique does not allow separation of individual DNA-EBC bands). To determine the components of a band, the band must be cut from many DNA-EBC gels, the proteins eluted and concentrated, subjected to SDS-PAGE, and then hybridized with antibodies specific for each protein identified by mass spectrometry. Once this is complete, it will be possible to rapidly compare purified DNA-EBC proteins from different cell lines (e.g., DNA repair mutants of various sorts) by simple SDS-PAGE of bead-purified complexes. Also, purified proteins can be further analyzed to detect specific post-translational modifications (e.g., cells can be labeled with ³²P-P0₄, and the phosphorylation patterns of DNA-EBC proteins determined in, for example, DNA-PK mutants). The pattern of proteins within the DNA-EBC can even be compared with the general pool of nuclear proteins so that the effects of various modifications leading to DNA-EBC incorporation can be studied.

### EXAMPLE 10

### DNA-EBC Pattern Can Predict Tumor Radiosensitivity

Predicting response of tumors to radiation is as important as predicting toxicity. Preliminary data from several tumor cell lines demonstrated a DNA-EBC pattern similar to that of primary fibroblasts. Since SF2 of tumor cells can predict tumor radiocurability, it is reasonable to test whether DNA-EBC pattern also predicts radiocurability.

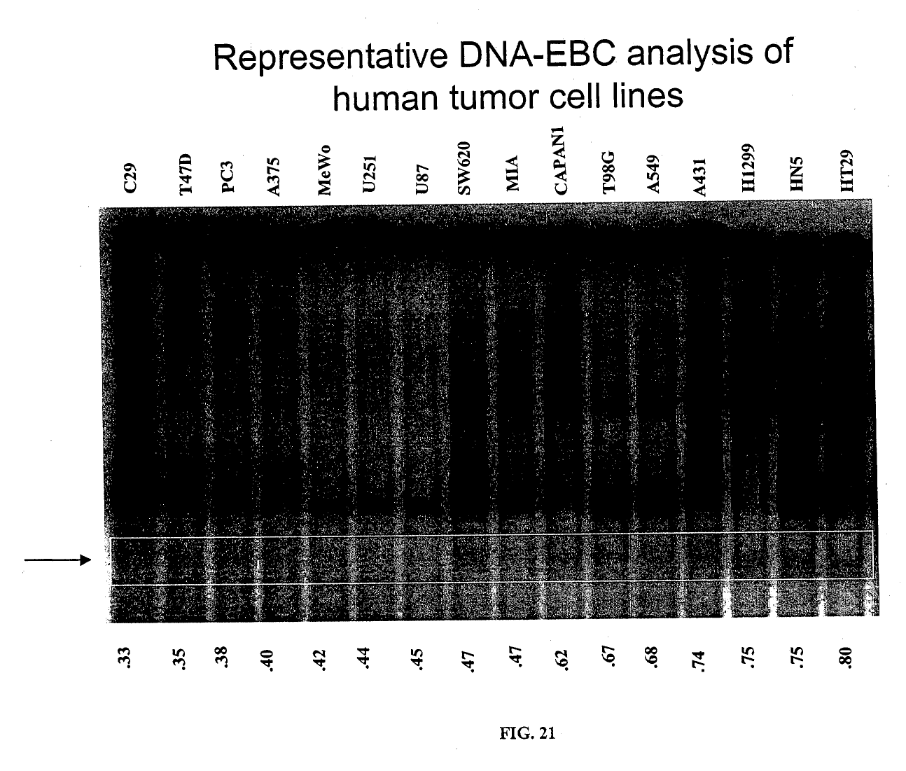

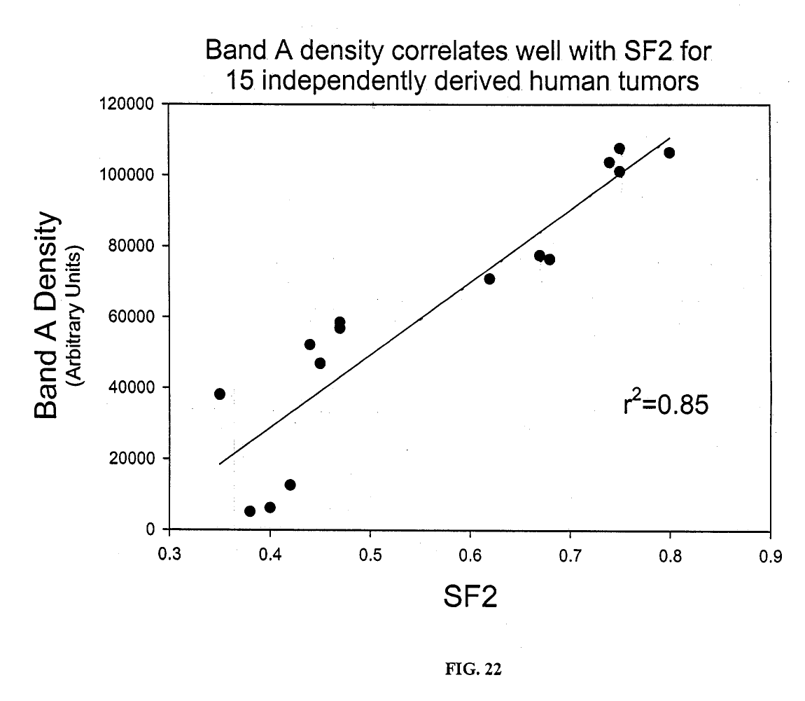

Thus, the DNA-EBC banding pattern/band density of a variety of human tumors with a spectrum of radiosensitivities was determined. A majority of the normal tissue specimens were generated from head & neck cancer patients Other cell lines from cervical cancers, lung cancers and melanomas with different SF2s were also obtained from commercial sources and their DNA-EBC patterns determined. FIGS. 21 and 22 provide results which support the prediction of radiosensitivity in a variety of tumor cells. FIG. 21 shows DNA-EBC analysis of these tumor cells. FIG. 22 demonstrates that band A density correlates well with SF2 for independently derived human tumors.

All of the methods disclosed and claimed herein can be made and executed without undue experimentation in light of the present disclosure. While the methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.

U.S. Pat. No. 4,704,362

U.S. Pat. No. 5,221,619

U.S. Pat. No. 5,583,013

U.S. Pat. No. 4,659,774

U.S. Pat. No. 4,816,571

U.S. Pat. No. 5,141,813

U.S. Pat. No. 5,264,566

U.S. Pat. No. 4,959,463

U.S. Pat. No. 5,428,148

U.S. Pat. No. 5,554,744

U.S. Pat. No. 5,574,146

U.S. Pat. No. 5,602,244

Achary et al., Cytogenet. Cell Genet., 91(1-4):39-43, 2000.

Aprelikova et al., Proc. Natl Acad. Sci. USA, 96(21):11866-11871, 1999.

Baskaran et al., Nature, 387:516-519, 1997.

Beaucage and Lyer, Tetrahedron, 48:2223-2311, 1992.

Berwick and Vineis, J. Natl. Cancer Inst., 92:874-897, 2000.

Bhatia et al., N. Engl. J. Med., 334(12):745-51, 1996.

Bischof et al., J. Cell Biol., 153(2):367-380, 2001.

Bjork-Eriksson et al., Brit. J. Cancer, 80(9):1400-1404, 1999a.

Bjork-Eriksson et al., Int. J. Radiat. Oncol. Biol. Pizys., 45(4):1005-1010, 1999.

Bjork-Eriksson et al., Int. J. Radiat. Oncol. Biol. Phys., 46(1):13-19, 2000.

Blocher et al., Int. J. Radiat. Biol., 60:791-802, 1991.

Brock et al., Int. J. Radiat. Oncol. Biol. Phys., 32(5):1371-1379, 1995.

Brock et al., Int. J. Radiat. Oncol. Biol. Phys., 32:1371-1379, 1995.

Buchholz and Wu, Int. J. Radiat. Oncol. Biol. Phys., 49(2):533-537, 2001.

Buchholz et al., Int. J. Cancer, 2001.

Buchholz et al., Int. J. Cancer, 97(5):557-561, 2002.

Buffa et al., Int. J. Radiat. Oncol. Biol. Phys., 50(5):1113-1122, 2001.

Burke et al., JAMA, 277:997-1003, 1997.

Burnet et al., Int. J. Radiat. Oncol. Biol. Phys. 70:289-300, 1996.

Burnet et al., Lancet, 339:1570-1571, 1992.

Burnet et al., Radiother. Oncol., 33:228-238, 1994.

Calsou et al., J. Biol. Chem., 274(12):7848-7856, 1999.

Chakraborty and Sankaranarayanan, Genet. Res., 72(3):191-198, 1998.

Chen et al., Mol. Cell, 2(3):317-328, 1998.

Collins et al., Oncogene, 10: 1673-1675, 1995.

Cortez et al., Science, 286:1162-1166, 1999.

Davies et al., Mol. Cell, 7(2):273-282, 2001.

DeFazio et al., EMBO. J, 21(12):3192-3200, 2002.

Dyan and Yoo, Nucleic Acids Res., 26:1551-1559, 1998.

Geara et al., Int. J. Radiat. Oncol. Biol. Phys., 27:1173-1179, 1993.

Getts and Stamato, J. Biol. Chem., 269(23):15981-1594, 1994.

Gillam et al., J. Biol. Chem., 253(8):2532-2539, 1978.

Gilliam and Smith, Gene, 8(1):81-97, 1979.

Gowen et al., Science, 281:1009-1012, 1998.

Grawunder et al., J. Biol. Chem., 273(38):24708-24714, 1998.

Hannan et al., Cancer Res., 61(9):3627-3631, 2001.

Hart et al., Int. J. Radiat. Oncol. Biol. Phys., 13(8):1237-1240, 1987.

Hayman et al., J. Clin. Oncol., 19(1):127-136, 2001.

Helzlsouer et al., J. Natl. Cancer Inst., 88(11):754-5, 1996.

Hsu et al., Int. J. Cancer, 15;43(3):403-409, 1989.

Itakura and Riggs, Science, 209(4463):1401-1405, 1980.

Itakura et al., J. Am. Chem. Soc., 97(25):7327-7332, 1975.

Johansen et al., Radiother. Oncol., 40:101-109, 1996.

Johansen et al., Radiother. Oncol., 40:101-109, 1996.

Khorana, Science, 203(4381):614-625, 1979.

Kishi et al., Cancer Res., 60(5):1326-1331, 2000.

Lewis et al., Int. J. Radiat. Oncol. Biol. Phys. 34(1):103-110, 1996.

Mahajan et al., Mol. Cell Biol., 22(14):5194-5202, 2002.

Mattsson et al., Br. J. Cancer, 82(1):220-226, 2000.

McKay and Hendry, Radiother. Oncol., 50:67-75, 1999.

McKay et al., Radiother. Oncol., 46:215-216, 1998.

Mohrenweiser and Jones, Mutat. Res., 400:3-20, 1999.

Moller et al., Cancer Epidemiol. Biomarkers Prev., 9(10):1005-1015, 2000.

Muller et al., Radiat. Environ. Biophy., 40(1):83-89, 2001.

Nimura et al., Radiat. Res., 157:562-567, 2002.

Norman et al., Am. J. Clin. Oncol., 11:84-88, 1988.

Okayasu et al., Cancer. Res., 60(16):4342-4325, 2000.

Parshad et al., Br. J. Cancer, 74(1):1-5, 1996.

Pataer et al., Cancer Res., 62(8):2239-2243, 2002.

Paull et al., Curr. Biol., 10(15):886-895, 2000.

Patel et al., Int. J. Cancer, 73(1):20-24, 1997.

Peters and McKay, Int. J. Radiother. Oncol. Biol. Phys., 49:501-504, 2001.

Savelyeva et al., Cancer Res., 61(13):5179-5185, 2001.

Scott, et al., Int. J. Radiat. Biol., 75(1):1-10, 1999.

Scully et al., Cell, 88:265-275, 1997.

Setlow, In: Human Carcinogenesis, Harris and Autrup (Eds.), Academic Press, 231-254, NY, 1983.

Sharan et al., Nature, 386:804-810, 1997.

Shen et al., Cancer Res., 58:604-608, 1998.

Smith et al., Nat. Genet., 2:128-131, 1992.

Staff et al., Genes Chromosomes Cancer, 28(4):432-442, 2000.

Stausbol-Gron and Overgaard, Radiother. Oncol., 50:47-55, 1999.

Stevens et al., Radiation Res., 252:25-30, 1999.

Taghian et al., Radiat., Res. 134(1):16-21, 1993.

Thames et al. Int. J. Radiat. Oncol. Biol. Phys., 22:241-246, 1992.

Tucker et al., Radiother. Oncol., 38:103-113, 1996.

Wang et al., Genes Dev., 14:927-939, 2000.

West and Hendry, Br. J. Radiol., 24(146-152, 1992.

West and Hendry, Br. J. Radiol., 24:146-152, 1992.

Yu et al., Cancer Res., 61(5):1820-1824, 2001.

Zhang et al., Int. J. Oncol., 17(2):399-404, 2000.

Zhao et al., Nature, 405(6785):473-477, 2000.

Zhong et al., Science, 285:747-750, 1999.

What is claimed is:

1. A method of assessing the susceptibility of a cell to DNA damage comprising the steps of:
(a) providing an extract comprising proteins from said cell;
(b) mixing said extract with a labeled oligonucleotide and an excess of non-labeled DNA;
(c) subjecting the mixture of step (b) to electrophoretic separation;
(d) determining the band shift of said labeled oligonucleotide; and
(e) comparing the band shift of said labeled oligonucleotide with that observed when a control is used,
wherein a change in said band shift, as compared to the control, indicates an altered sensitivity to DNA damage.

2. The method of claim 1, wherein said cell is isolated from a subject with cancer.

3. The method of claim 1, wherein said cell is isolated from a subject that does not have cancer.

4. The method of claim 1, wherein said cell is a primary fibroblast cell.

5. The method of claim 1, wherein said cell is a lymphocyte.

6. The method of claim 1, wherein said cell is obtained from a blood or tissue sample.

7. The method of claim 1, wherein said label comprises a radiolabel, a fluorescence label, a dye or an enzyme.

8. The method of claim 1, wherein said oligonucleotide is a radiolabeled oligonucleotide.

9. The method of claim 8, wherein said oligonucleotide is radiolabeled with ³²P.

10. The method of claim 8, wherein said radiolabeled oligonucleotide is end-labeled.

11. The method of claim 1, wherein said non-labeled DNA is supercoiled DNA.

12. The method of claim 1, wherein said control comprises proteins from a radiosensitive cell and/or a non-radiosensitive cell.

13. The method of claim 1, wherein said electrophoretic separation is carried out in 5% acrylamide gel under non-denaturing conditions.

14. The method of claim 1, wherein said proteins are nuclear extract proteins.

15. The method of claim 1, wherein said susceptibility is to radiation-induced DNA damage.

16. The method of claim 15, wherein the radiation is ionizing irradiation.

17. The method of claim 1, wherein said susceptibility is to chemical-induced DNA damage.

18. The method of claim 17, wherein the chemical is selected from the group consisting of cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate.

19. A method of predicting the risk of cancer in an individual comprising the steps of:
(a) providing an extract comprising proteins from cells of said individual;
(b) mixing said extract with a labeled oligonucleotide and excess of non-labeled DNA;
(c) subjecting the mixture of step (b) to electrophoretic separation;
(d) determining the band shift of said labeled oligonucleotide; and
(e) comparing the band shift of said labeled oligonucleotide with that observed when a control is used,
wherein a change in said band shift indicates altered susceptibility to DNA damage, which predicts altered risk of cancer in said individual.

20. A method of predicting the presence of BRCA mutations in an individual comprising:
(a) providing an extract comprising proteins from cells of said individual;
(b) mixing said extract with a labeled oligonucleotide and excess of non-labeled DNA;
(c) subjecting the mixture of step (b) to electrophoretic separation;
(d) determining the band shift of said labeled oligonucleotide; and
(e) comparing the band shift of said labeled oligonucleotide with that observed when a control is used,
wherein a decrease in band shift indicates BRCA mutations damage, which predicts an increased risk of breast cancer in said individual.

21. A method of predicting the toxicity of a DNA damaging cancer therapy comprising:
(a) providing an extract comprising proteins from cells of an individual;
(b) mixing said extract with a labeled oligonucleotide and excess of non-labeled DNA;
(c) subjecting the mixture of step (b) to electrophoretic separation;
(d) determining the band shift of said labeled oligonucleotide; and
(e) comparing the band shift of said labeled oligonucleotide with that observed when a control is used,
wherein the relative decrease in band shift is indicative of the relative therapeutic toxicity.

22. A method of measuring tumor cell sensitivity to a DNA damaging cancer therapy comprising:
(a) providing an extract comprising proteins from tumor cells;
(b) mixing said extract with a labeled oligonucleotide and excess of non-labeled DNA;
(c) subjecting the mixture of step (b) to electrophoretic separation;
(d) determining the band shift of said labeled oligonucleotide; and
(e) comparing the band shift of said labeled oligonucleotide with that observed when a control is used,
wherein a decrease in band shift is indicative of said cells being sensitive to said DNA damaging cancer therapy.
